(19) United States
(12) Patent Application Publication
(10) Pub. No.: US 2024/0316516 A1
(43) Pub. Date: Sep. 26, 2024

(54) DRYING OF BIOLOGICAL MATERIAL

(71) Applicant: CELEVENTUS AB, Solna (SE)

(72) Inventor: Fernando Acevedo Fonseca, Solna (SE)

(21) Appl. No.: 18/263,213

(22) PCT Filed: Feb. 2, 2022
(86) PCT No.: PCT/SE2022/050110

§ 371 Date: Jul. 27, 2023

(30) Foreign Application Priority Data
Feb. 2, 2021 (SE) 2150117-6

(51) Int. Cl. B01J 2/04; B01L 5/00

(52) U.S. Cl. CPC B01J 2/04; B01L 5/00; B01L 2300/0681; B01L 2300/069

(57) ABSTRACT

There is provided a method of drying a biological material, comprising the steps of: a) generating a flow of microdroplets of the biological material having a dry matter content below 25% (weight/volume); b) contacting the microdroplets with a gas flow, wherein the ratio of the gas flow to the flow of microdroplets is at least 300.000:1, preferably at least 600.000:1, more preferably at least 800.000:1, thereby drying the biological material to form particles; c) separating the particles from the gas flow; d) drying the gas flow from step c); and e) recirculating the dried gas flow from step d) to step b). A corresponding apparatus is also provided.

### TECHNICAL FIELD

The present disclosure relates to the field of drying biological material.

### BACKGROUND

The isolation and preservation of biological active structures has been an important field of research and development for the last decades in the medical, pharmaceutical, and industrial field as well as in many other areas.

There are various methods to preserve the biological activity of biological material.

Quick freezing biological structures at extremely low temperatures (liquid Nitrogen) has been a successful approach, particularly in cell preservation. The freezing process must be carried out at such speed that the structures do not have time enough to change. Keeping deep-frozen biological systems is resource consuming.

Removal of water while the structures are in the frozen stadium, known as “freeze-drying”, is a slow and time-consuming process. Complex biological structures can lose the activity upon freeze-drying.

Another approach to preserve the biological activity is spray drying, which basically consists of spraying the material to be dried into tiny droplets. Since the 1940s, spray drying has been used successfully in the pharmaceutical industry to produce temperature-insensitive drug substances and various excipients, such as analgesics, antibiotics, vitamins, and antacids.

The use of high temperatures, often well over 100° C. is generally used to spray dry at a reasonable speed, renders the technique less usable for drying of temperature-sensitive materials. Since late 1950s, spray-drying encapsulation has been used in the food industry for protection from degradation and oxidation of oil flavor and production of powders from liquids. Spray drying is however considered inappropriate for drying heat-sensitive biological materials, such as many pharmaceutical proteins and enzymes (Ida I. Muhamad et al. in Ingredients Extraction by Physicochemical Methods in Food, 2017).

### SUMMARY

An objective of the present disclosure is to provide a method and an apparatus for drying biological material at a high rate. Another objective is to maintain the activity of the biological material during drying. Yet another objective is to minimize the losses of biological material during drying.

Activity loss of biological systems can be triggered by changes in the water environment that generate changes in the structure of the system. These structural changes usually take time to happen.

Drying of a biological structure implies a radical change in its water environment. However, if the drying happens in shorter time than the time for the structural changes take place, it is possible to keep the biological activity in the dried product.

If the drying medium (gas) to water ratio is sufficiently large and the droplets of the aqueous biological material are sufficiently small, a quick drying process that preserves biological activity can be obtained.

Accordingly, the following itemized listing of embodiments of the present disclosure is provided:

1. A method of drying a biological material, comprising the steps of:
a) generating a flow of microdroplets of the biological material having a dry matter content below 25% (weight/volume), preferably below 20% (weight/volume), such as below 15% (weight/volume), such as below 10% (weight/volume);
b) contacting the microdroplets with a gas flow, such as an airflow, wherein the ratio of the gas flow to the flow of microdroplets is at least 300,000:1, preferably at least 600,000:1, more preferably at least 800,000:1, thereby drying the biological material to form particles;
c) separating the particles from the gas flow;
d) drying the gas flow from step c); and
e) recirculating the dried gas flow from step d) to step b).

2. The method of item 1, wherein the average diameter of the microdroplets generated in step a) is below 10 μm, preferably below 5 μm.

3. The method of item 1 or 2, wherein the flow of microdroplets are generated by means of at least one nebulizer, such as at least one ultrasonic mesh nebulizer, in step a).

4. The method of any one of the preceding items, wherein in step b), the flow of microdroplets is supplied to the gas flow at an angle to the direction of the gas flow.

5. The method of item 4, wherein the angle is 45°-135°, preferably 75°-105°.

6. The method of item 4 or 5, wherein the direction of the gas flow is essentially horizontal.

7. The method of any one of the preceding items, wherein the average residence time between the first contact between the microdroplets and the gas flow in step b) and the separating of step c) is at least 0.2 s, such as 0.5-5.0 s, such as 0.5-3.0 s.

8. The method of any one of the preceding items, wherein the rate of the gas flow is at least 1.0 m³/min, preferably at least 2.0 m³/min.

9. The method of any one of the preceding items, wherein the flow of microdroplets is generated in step a) at a rate of 50-2000 ml/h, such as 100-1000 ml/h.

10. The method of any one of the preceding items, wherein step d) comprises cooling the gas flow and/or contacting the gas flow with silica gel.

11. The method of any one of the preceding items, wherein step d) comprises condensation of water in the gas flow.

12. The method of any one of the preceding items, wherein the gas flow is filtrated at the end of step d).

13. The method of any one of the preceding items, wherein the gas flow supplied to step d) has a relative humidity below 30%, preferably below 25%.

14. The method of any one of the preceding items, wherein step c) comprises filtering of the gas flow, e.g. using a mesh filter, such as a nylon mesh filter.

15. The method of item 14, wherein step c) comprises at least two consecutive filtering steps.

16. The method of any one of items 1-13, wherein step c) comprises cyclonic separation of particles from the gas flow.

17. The method of item 16, wherein the gas flow from the cyclonic separation is filtered.

18. The method of any one of the preceding items, wherein the biological material is selected from the group consisting of peptides, proteins, vaccines, inactivated or attenuated viruses and cellular microstructures.

19. An apparatus for a drying biological material, comprising:
i) at least one nebulizer for generating a flow of microdroplets of the biological material having a dry matter content below 25% (weight/volume), preferably below 20% (weight/volume), such as below 15% (weight/volume), such as below 10% (weight/volume);
ii) a fan or a pump for generating a gas flow;
iii) a compartment for contacting the microdroplets generated by the at least one nebulizer with the gas flow and thereby drying the biological material to form particles;
iv) a separation arrangement for separating particles formed in the compartment from the gas flow;
v) a drying arrangement for drying the gas flow from the separation arrangement;
and
vi) means for recirculating the dried gas flow from the drying arrangement to the compartment.

20. The apparatus of item 19, wherein the separation arrangement comprises a filter, such as a mesh filter.

21. The apparatus of item 20, wherein the separation arrangement further comprises a second filter, which preferably is a particle-absorbing filter.

22. The apparatus of item 19, wherein the separation arrangement comprises a cyclone separator.

23. The apparatus of item 20, wherein the separation arrangement further comprises a filter, which preferably is a particle-absorbing filter, arranged downstream the cyclone separator.

24. The apparatus of any one of items 19-23, wherein the at least one nebulizer is at least one mesh nebulizer, such as at least one piezoelectric mesh nebulizer.

25. The apparatus of any one of items 19-24, wherein the compartment comprises a microdroplets inlet for supplying microdroplets to the gas flow at an angle to the direction of the gas flow.

26. The apparatus of item 25, wherein the angle is 45°-135°, preferably 75°-105°.

27. The apparatus of any one of items 19-26, wherein the compartment is configured to give the gas flow an essentially horizontal direction through it.

28. The apparatus of any one of items 19-27, wherein the drying arrangement comprises silica gel.

29. The apparatus of any one of items 19-28, wherein the drying arrangement comprises a cooling element for condensing water in the gas flow.

30. The apparatus of any one of items 19-29, wherein the drying arrangement comprises a filter for filtering the gas flow after drying.

31. The apparatus of any one of items 19-30, further comprising at least one sensor for sensing a pressure, temperature and/or relative humidity of the gas flow, which at least one sensor is connected to a central processing unit arranged to control the pump/fan and/or the at least one nebulizer in response to (a) signal(s) from the at least one sensor.

### BRIEF DESCRIPTION OF THE DRAWINGS

FIG. 1 illustrates an embodiment 100 of the apparatus of the present disclosure.

### DETAILED DESCRIPTION

As a first aspect of the present disclosure there is provided a method of drying a biological material. The biological material may be selected from the group consisting of peptides, proteins, vaccines (including RNA vaccines), inactivated or attenuated viruses and cellular microstructures. The peptides may be selected from the group consisting of hormones, cofactors, antibiotic peptides and bioactive peptides. The proteins may be selected from the group consisting of enzymes, immunoglobulins and plasma components.

The method is typically used in the medical/pharmaceutical field, but may also be used in a larger/industrial scale for the production of enzymes, e.g. selected from the group consisting of peroxidases, catalases, peptidases and amylases.

The method comprises the step of:
a) generating a flow of microdroplets of the biological material having a dry matter content below 25% (weight/volume). Preferably, the dry matter content of the generated microdroplets is below 20% (weight/volume), such as below 15% (weight/volume), such as below 10% (weight/volume).

To facilitate an efficient drying, the average diameter of the microdroplets generated in step a) is typically small, preferably below 10 μm, more preferably below 5 μm.

The flow of microdroplets may be generated in step a) by means of at least one nebulizer, such as at least one ultrasonic nebulizer or at least one jet nebulizer. The at least one ultrasonic nebulizer may be at least one mesh nebulizer, such as at least one piezoelectric mesh nebulizer. This is further discussed below in connection with the second aspect.

In step a), the flow of microdroplets is typically generated at a rate of at least 50 ml/h, such as at least 100 ml/h. A typical upper limit may be 1000 or 2000 ml/h. The higher rates typically require several nebulizers.

The method further comprises the step of:
b) contacting the microdroplets with a gas flow, thereby drying the biological material to form particles.

The gas flow is preferably an airflow or a flow of nitrogen. The latter may be more preferred in case of an oxygen-sensitive biological material.

The contact of step b) is a direct contact. Hence the gas flow is not separated from the microdroplets by a membrane or another type of barrier. Step b) is typically carried out in a drying chamber. This chamber is preferably not heated.

To facilitate an efficient, yet gentle drying, the ratio of the gas flow to the flow of microdroplets in step b) is at least 300,000:1, preferably at least 600,000:1, more preferably at least 800,000:1. As understood by the skilled person, the flows referred to here are volumetric flow rates.

To obtain a satisfactory drying rate, the rate of the gas flow is preferably at least 1.0 m³/min, such as at least 2.0 m³/min. A typical upper limit for the gas flow may be 4 m³/min.

In step b), the flow of microdroplets is preferably supplied to the gas flow at an angle to the direction of the gas flow. It is thus preferred that the flow of microdroplets is non-linear with respect to the gas flow. The angle may be in the range of 45°-135°, preferably 75°-105°. In one embodiment, the angle is about 90°.

In one embodiment, the direction of the gas flow is essentially horizontal.

The method further comprises the step of:
c) separating the particles from the gas flow.

The average residence time between the first contact between the microdroplets and the gas flow in step b) and the separating of step c) is normally at least 0.2 s, preferably 0.5-5.0 s, such as 0.5-3.0 s. A longer time would require an unnecessary long drying chamber.

In an embodiment, step c) comprises filtering of the gas flow, e.g. using a mesh filter, such as a nylon mesh filter. Suitable filters are discussed below in connection to the second aspect.

Step c) may comprise at least two consecutive filtering steps, wherein a particle absorbing filter, such as a HEPA filter, is used for the last filtering step. The purpose of such an absorbing filter is to prevent leakage of biological material.

In an alternative or complimentary embodiment, step c) comprises cyclonic separation of particles from the gas flow. The gas flow from the cyclonic separation may be filtered in at least one step. Again, a particle absorbing filter, such as a HEPA filter, is used for the last filtering step.

The method further comprises the step of:
d) drying the gas flow from step c).

In an embodiment, step d) comprises contacting the gas flow with silica gel or molecular sieves. Silica gel may be more preferred due to the high temperatures needed to regenerate the molecular sieve.

In an alternative or complimentary embodiment, step d) comprises condensation of water in the gas flow, typically by contacting the gas flow with a cooling element (further discussed below). The gas flow is typically subjected to pumping between steps c) and d). Thereby the cooling action of the drying step counteracts the temperature increase caused by said pumping, Accordingly, the temperature is reduced before the gas flow is recirculated (see step e) below) and contacts the biological material.

In a preferred embodiment, step d) comprises condensation of water in the gas flow followed by contacting the gas flow with silica gel or molecular sieves. The method further comprises the step of:
e) recirculating the dried gas flow from step d) to step b).

Consequently, the gas used to dry biological material is reused after the “regeneration” of step d), meaning that gas circulates in the method of the first aspect. Preferably, the method comprises sensing the relative humidity of the gas flow in at least one of the steps and controlling the method in response thereto. As an example, the rate of the flow of microdroplets generated in step a) may be reduced if such sensing indicates a decreased drying capacity in step d). A relative humidity above a predetermined reference value is a typical indication of a decreased drying capacity.

Preferably, the method is controlled to maintain the relative humidity of the gas flow supplied to step d) below 30%, more preferably below 25%. Further, the method is preferably controlled to maintain relative humidity of the gas flow supplied to step b) below 15%, more preferably below 10%.

Further, the temperature of the gas flow in the method is preferably kept below 30° C., preferably below 25° C.

To prevent that particulate material from the drying step contaminates the biological material, the gas flow may be filtrated at the end of step d) or at least before being recirculated to step b).

As a second aspect of the present disclosure, there is provided an apparatus for a drying biological material. Examples of biological materials are discussed above. The apparatus of the second aspect is suitable for carrying out the method of the first aspect.

The apparatus comprises:
i) at least one nebulizer for generating a flow of microdroplets of the biological material having a dry matter content below 25% (weight/volume), preferably below 20% (weight/volume), such as below 15% (weight/volume), such as below 10% (weight/volume).

In an embodiment, the at least one nebulizer is at least one jet nebulizer. In another embodiment, the at least one nebulizer is at least one ultrasonic nebulizer, such as at least one mesh nebulizer, such as at least one piezoelectric mesh nebulizer. To generate a higher flow, more than one nebulizer is typically needed.

The apparatus further comprises:
ii) a fan or a pump for generating a gas flow, such as a gas flow of at least 1.0 m³/min, such as at least 2.0 m³/min. The gas flow may for example be an airflow or a flow of nitrogen. An example of a suitable fan is given in the EXAMPLE section below.

The apparatus further comprises:
iii) a compartment for contacting the microdroplets generated by the at least one nebulizer with the gas flow and thereby drying the biological material to form particles.

The compartment is designed for a direct contact between the microdroplets and the gas flow. Preferably, no heater is arranged to heat the gas flow in the compartment.

The compartment may comprise a microdroplets inlet for supplying microdroplets to the gas flow at an angle to the direction of the gas flow. The angle may be 45°-135°, preferably 75°-105°, such as about 90°.

In one embodiment, the compartment is configured to give the gas flow an essentially horizontal direction through it.

The apparatus further comprises:
iv) a separation arrangement for separating particles formed in the compartment from the gas flow.

In an embodiment, the separation arrangement comprises a filter, such as a mesh filter. An example of a suitable mesh filter is given in the EXAMPLE section below.

In a further embodiment, the separation arrangement comprises at least two filters. In such case, the last filter may be a particle-absorbing filter, such as a HEPA filter. In one embodiment, the separation arrangement comprises a first particle-separating filter, a second particle-separating filter and a particle-absorbing filter arranged in series.

In an alternative or complementary embodiment, the separation arrangement comprises a cyclone separator. A particle-absorbing filter may be arranged downstream such a cyclone separator.

In one embodiment, the separation arrangement comprises cyclone separator, a particle-separating filter and a particle-absorbing filter arranged in series. In this case, particles may be recovered from the cyclone and the particle-absorbing filter.

The apparatus further comprises:
v) a drying arrangement for drying the gas flow from the separation arrangement.

In one embodiment, the drying arrangement comprises silica gel or molecular sieves, preferably silica gel.

In an alternative or complementary embodiment, the drying arrangement comprises a cooling element for condensing water in the gas flow.

As an example, the drying arrangement may comprise the cooling element arranged upstream the silica gel or molecular sieves.

An embodiment of the drying arrangement comprises a filter for filtering the gas flow after drying. Alternatively, this filter may be arranged at the inlet of the compartment. The filter is typically an absorbing filter, such as a HEPA filter.

The apparatus further comprises:
vi) means for recirculating the dried gas flow from the drying arrangement to the compartment.

The fan or pump is preferably arranged between the separation arrangement and the drying arrangement in the sense that an outlet of the separation arrangement is connected to an inlet of the fan or pump and an outlet of the fan or pump is connected to an inlet of the drying arrangement.

An embodiment of the apparatus further comprises at least one sensor for sensing a pressure, temperature and/or relative humidity of the gas flow, which at least one sensor is connected to a central processing unit arranged to control the pump/fan and/or the at least one nebulizer in response to (a) signal(s) from the at least one sensor. Examples of this embodiment is further discussed in the EXAMPLE section below.

### EXAMPLE

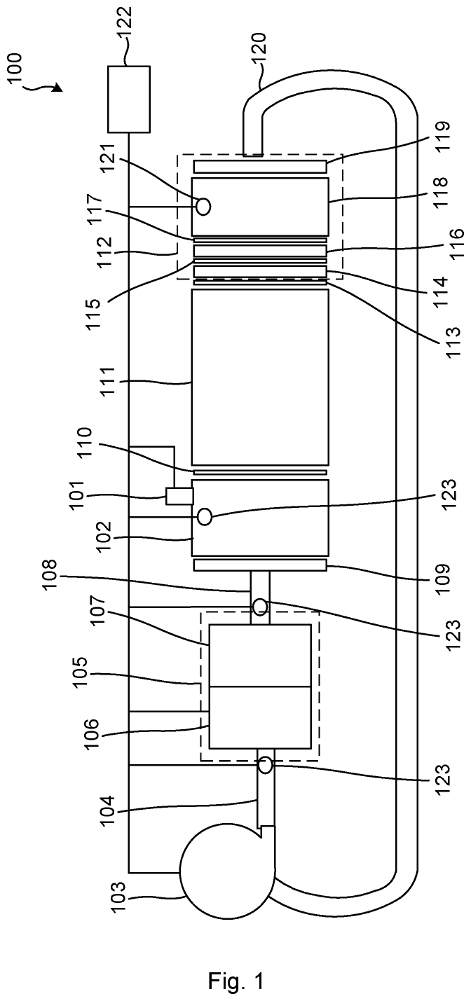

An embodiment 100 of the apparatus of the present disclosure is illustrated in FIG. 1. The embodiment 100 comprises four piezoelectric mesh nebulizers 101 for generating a flow of microdroplets of a biological material. Each nebulizer suitably has the following characteristics: 20 mm diameter; 2.5 W; 113 kHz; 0.5-1.0 mL/min). The total flow from the nebulizers 101 is thus 2-4 mL/min. The nebulizers 101 are arranged in the upper part of a nebulizing chamber 102.

The embodiment 100 further comprises a radial fan 103 (1 or 2 BHP) capable of generating a horizontal airflow of 2-3 m³/min.

An air channel 104 connects an outlet of the fan 103 to an inlet of a drying arrangement 105 comprising a condensing unit 106 (for condensing water in the airflow) followed by an absorption unit 107 comprising silica gel. The condensing unit 106 preferably comprises a water-cooled cooling element. The condensing unit has a dual purpose; it reduces the relative humidity of the airflow and cools the airflow, which facilitates the downstream silica gel absorption and protects heat-sensitive biological material. During operation, the drying arrangement typically reduces the relative humidity of the airflow from about 20% to about 10%. In the beginning of a run, the relative humidities are lower and then gradually increases during the run. When the relative humidities increase to a certain level, reflecting a reduced drying capacity of the drying arrangement 105, the run is typically stopped and the silica gel is regenerated.

Another air channel 108 connects an outlet of the drying arrangement to an inlet filter 109 arranged at the nebulizing chamber 102. The inlet filter 109 is an absorbing (particulate arresting) filter preventing contamination of the biological material.

In the nebulizing chamber 102, the flow of microdroplets is supplied to the airflow at an angle of about 90° to the direction of the airflow.

An outlet of the nebulizing chamber 102 is sealed 110 to an inlet of a drying chamber 111, e.g. by silicone sealing. Collectively, the nebulizing chamber 102 and the drying chamber 111 form a compartment for contacting the microdroplets generated by the nebulizers 101 with the airflow and thereby drying the biological material to form particles. A reason for separating the compartment into two separate units is to facilitate cleaning and modification of the respective chambers.

A separation arrangement 112 for separating particles formed in the compartment from the airflow is sealed 113 to an outlet of the drying chamber 111, e.g. by silicon sealing. The separation arrangement comprises a first particle-separating filter 114, which is a nylon mesh filter (400 Mesh). Downstream the first particle-separating filter 114, a second particle-separating filter may be arranged in a separate frame (not shown). The pore size of the second particle-separating filter may be 0.5-10 microns, such as 1-6 microns. The first and (when included) the second particle-separating filter is/are sealed 115 to a filter support 116, which in turn is sealed 117 to an inlet of a filter chamber 118. The purpose of the filter chamber 118 is to obtain an even distribution of the airflow. The shape of the filter chamber 118 may be conical (not shown). At the outlet of the filter chamber 118, an absorbing (particulate arresting) filter 119 is arranged to prevent leakage of particulate/biological material.

Yet another air channel 120 connects an outlet of the separation arrangement 112 to an inlet of the fan 103, thereby facilitating recirculation of the airflow.

A filter chamber sensor 121 is arranged in the filter chamber 118 to sense the pressure, relative humidity and temperature of the airflow therein. The filter chamber sensor is in connection with a central processing unit 122 that configured to receive a signal from the filter chamber sensor 121 and generate a control signal in response thereto. The control signal may be received by the nebulizers 101 and/or the fan 103. The flow rate of the microdroplets may be adjusted in response to the control signal. As an example, this flow rate may be reduced if the filter chamber sensor 121 has sensed a relative humidity above a reference value. Further, the fan 103 may be controlled in response to the control signal. As an example, the fan 103 and the nebulizers 101 may be turned off if the filter chamber sensor 121 has sensed a pressure below a reference above or a temperature above a reference value. The control signal may also be based on signals from further sensors 123 arranged in the apparatus 100.

A sample of 5 g commercial a-amylase (molecular weight ˜58 KDa, commonly used for wine clarification) and 15 g trehalose was dissolved in 150 ml phosphate saline buffer (PBS). A 5 ml aliquot was kept for reference activity measurements (reference sample). The rest of solution was dried using an apparatus according to the embodiment 100 described above. The dried and recovered powder represented >80% of the solids at start. A sample of the dried material was diluted in water to match the concentration of the reference sample. Enzyme activates were tested by incubating the enzyme solutions (processed sample and reference sample) as well as a blank and a positive control, with aliquots of a starch solution in PBS at room temperature. Samples of the incubation mixtures were taken at exact time intervals of 1 min and tested with Lugol reagent to assess the end of the starch hydrolysis. Both enzyme solutions gave same time result for the complete starch hydrolysis. This shows that the enzymatic activity was preserved in the drying process.

One capsule of commercial lactase (molecular weight ˜160 KDa, commonly used for digestive purposes) 5000 FCC per capsule was dispersed in 10 mL PBS under shaking for 5 min. The suspension was then centrifuged at 4000 RPM for 4 min. A clear supernatant (about 8 mL) was collected, representing the enzyme extract. The extract was diluted to 120 mL with PBS and 10 g trehalose was added to the solution. A 5 ml aliquot was kept for start material activity measurements (reference sample). The rest of solution was dried using an apparatus according to the embodiment 100 described above. >80% of the solids in start material were collected from the filter. A sample of the dried material was diluted in water to match the concentration of the reference sample. Enzyme activities were tested by incubating the enzyme solutions (processed sample and reference sample) as well as a blank and a positive control, for different times with aliquots of a reagent solution containing 0-nitrophenyl-β-d-galactopyranoside. The incubation at room temperature was stopped by adding a solution of NaOH to aliquots taken at the time intervals of 1 min. The color development gave same time result for both samples, which shows that the enzymatic activity was preserved in the drying process.

1. A method of drying a biological material, comprising the steps of:
a) generating a flow of microdroplets of the biological material having an average diameter below 10 μm and a dry matter content below 25% (weight/volume), preferably below 20% (weight/volume), such as below 15% (weight/volume), such as below 10% (weight/volume);
b) contacting the microdroplets with a gas flow, such as an airflow, wherein the ratio of the gas flow to the flow of microdroplets is at least 300,000:1, preferably at least 600,000:1, more preferably at least 800,000:1, thereby drying the biological material to form particles;
c) separating the particles from the gas flow;
d) cooling and drying the gas flow from step c); and
e) recirculating the dried gas flow from step d) to step b).

2. The method of claim 1, wherein the average diameter of the microdroplets generated in step a) is below 5 μm.

3. The method of claim 1, wherein in step b), the flow of microdroplets is supplied to the gas flow at an angle to the direction of the gas flow, wherein said angle is preferably 45°-135°, more preferably 75°-105°.

4. The method of claim 3, wherein the direction of the gas flow is essentially horizontal.

5. The method of claim 1, wherein the average residence time between the first contact between the microdroplets and the gas flow in step b) and the separating of step c) is at least 0.2 s, such as 0.5-5.0 s, such as 0.5-3.0 s.

6. The method of claim 1, wherein the rate of the gas flow is at least 1.0 m³/min, preferably at least 2.0 m³/min.

7. The method of claim 1, wherein the flow of microdroplets is generated in step a) at a rate of 50-2000 ml/h.

8. The method of claim 1, wherein the gas flow supplied to step b) has a relative humidity below 15%, preferably below 10% and/or a temperature below 30° C., preferably below 25° C.

9. The method of claim 1, wherein step c) comprises filtering of the gas flow, e.g. using a mesh filter, such as a nylon mesh filter.

10. The method of claim 1, wherein the biological material is selected from the group consisting of peptides, proteins, vaccines, inactivated or attenuated viruses and cellular microstructures.

11. An apparatus for a drying biological material, comprising:
i) at least one nebulizer for generating a flow of microdroplets of the biological material having an average diameter below 10 μm and a dry matter content below 25% (weight/volume);
ii) a fan or a pump for generating a gas flow;
iii) a compartment for contacting the microdroplets generated by the at least one nebulizer with the gas flow and thereby drying the biological material to form particles;
iv) a separation arrangement for separating particles formed in the compartment from the gas flow;
v) a drying arrangement for drying the gas flow from the separation arrangement, which drying arrangement comprises a cooling element; and
vi) means for recirculating the dried gas flow from the drying arrangement to the compartment.

12. The apparatus of claim 11, wherein the separation arrangement comprises a filter, such as a mesh filter.

13. The apparatus of claim 11, wherein the at least one nebulizer is at least one mesh nebulizer, such as at least one piezoelectric mesh nebulizer.

14. The apparatus of claim 11, wherein the drying arrangement comprises silica gel.
